(19) United States
(12) Patent Application Publication
(10) Pub. No.: US 2003/0125243 A1
(43) Pub. Date: Jul. 3, 2003

(54) Synthesis of cyclic peptides

(76) Inventors: Jun Liu, Clarksville, MD (US); Xuedong Dai, San Jose, CA (US); Zhuang Su, Cambridge, MA (US)

Correspondence Address: Choate, Hall & Stewart, Exchange Place, 53 State Street, Boston, MA, 02109, US

(21) Appl. No.: 09/909,269

(22) Filed: Jul. 19, 2001

Related U.S. Application Data
(60) Provisional application No. 60/219,869, filed on Jul. 20, 2000.

(51) Int. Cl. A61K 38/12; C07K 7/64; C07K 5/12

(52) U.S. Cl. 514/9; 530/317

(57) ABSTRACT

The cyclic pentapeptide cyclo(-Arg-Gly-Asp-D-Phe-Lys-) is a highly potent and selective inhibitor for the αᵥβ₃ integrin. A related compound, cyclo(-Arg-Gly-Asp-D-Phe-Val-), is a promising anticancer drug candidate; it has been found to inhibit angiogenesis and induce apoptosis in vascular cells. We have developed a synthesis of arginine containing cyclic peptides using the cyclizcation reagent 1-propanephosphonic acid cyclic anhydride (T3P) and the guanidine protecting group, 2,2,5,7,8-pentamethylchroman-6-sulfonyl (Pbf). This improved synthesis is environmentally friendly and is generally applicable to the synthesis of other cyclic peptides.

### RELATED APPLICATIONS

The present application claims priority to provisional application U.S. Ser. No. 60/219,869, filed Jul. 20, 2000, which is incorporated herein by reference in its entirety.

### GOVERNMENT SUPPORT

The work described herein was supported, in part, by a grant from the National Institutes of Health (CA78743). The United States government may have certain rights in the invention.

### BACKGROUND OF THE INVENTION

As more and more peptides and proteins are identified and their role in the body is more clearly understood, they are being increasingly considered as therapeutic agents for use in the clinic. Proteins such as erythropoietin, insulin, and the interferons are just a few examples of proteins being used regularly in the clinic to treat disease, and many others are currently in clinical trials including the highly publicized anti-cancer drugs endostatin and angiostatin. However, the use of these highly specific and powerful agents as drugs has some major drawbacks including short half lives and the inability to deliver them orally due to proteolysis in the gastrointestinal tract.

In the search for peptides with longer half-lives in vivo, cyclic peptides have been found to possess an increased stability in the body when compared to the corresponding linear peptide. Nature has also used the cyclic peptide motif in designing antibiotics such as the polymixins, gramacidin S, and bacitracin. Another important class of cyclic peptides are the RGD-containing peptides that serve as antagonists of integrins.

The integrins are a class of cell surface adhesion proteins that play important roles in cell-cell and cell-matrix interactions (Integrins, Molecular and Biological Responses to the Extracellular Matrix; Cheresh, D. A., Mecham, R. P., Eds.; Academic Press: London, 1994; Cell Adhesion Molecules in Cancer and Inflammation; Epenetos, A., Pignatelli, M., Eds.; Harwood Academic Publishers: Chur, 1995; Hynes Cell 69:11-25, 1992; each of which is incorporated herein by reference). They are involved in diverse physiological processes. In particular, it has been shown that the αᵥβ₃ and αᵥβ₅ subgroups of integrins are required for tumor-induced angiogenesis. Inhibition of binding of αᵥβ₃ and αᵥβ₅ integrins to their native ligands by antibodies or cyclic peptides interferes with angiogenesis and induces tumor regression (Brooks et al. Cell 79:1157-1164, 1994; Brooks et al. Science 264:569-571, 1994; Friedlander et al. Science 270:1500-1502, 1995; Lode et al. Proc. Natl. Acad. Sci. USA 96: 1591-1596, 1999; each of which is incorporated herein by reference). Antagonists of αᵥβ₃ are undergoing clinical trials as potential cancer therapeutic agents (Brooks et al. Cell 79:1157-1164, 1994; incorporated herein by reference).

Integrins interact with their ligand proteins mainly through a tripeptide motif consisting of Arg-Gly-Asp (RGD). A number of RGD-containing peptides have been designed, synthesized, and tested for their ability to serve as antagonists of integrins. Of the antagonists synthesized to date (Haubner et al. J. Am. Chem. Soc. 118:7461-7472, 1996; Wermuth et al. J. Am. Chem. Soc. 199:1328-1335, 1997; Dechantsreiter et al. Med. Chem. 42:3033-3040, 1999; Haubner et al. J. Am. Chem. Soc. 118:7461-7472, 1996; Wermuth et al. J. Am. Chem. Soc. 199:1328-1335, 1997; Hammes et al. Nature Med.. 2:529-533, 1996; Wissmann et al. Phosphorus Sulfur 30:645-8, 1987; Wissmann et al. Angew. Chem. 92:129-130, 1980; Fields et al. Tetrahedron Lett. 34:6661, 1993; Carpino et al. Tetrahedron Lett. 34:7782, 1993; each of which is incorporated herein by reference), the cyclic peptide RGDfV (1, FIG. 1) (the lower case letter has been used to denote the D-configuration rather than the natural L-configuration) and its methylated analog made by Kessler and coworkers are the most selective for αᵥβ₃ (Haubner et al. Am. Chem. Soc. 118:7461-7472, 1996; Wermuth et al. J. Am. Chem. Soc. 199:1328-1335, 1997; each of which is incorporated herein by reference).

Another integrin antagonist is the cyclic pentapeptide cRGDfK (2, FIG. 1). First designed and synthesized by Kessler's group (Haubner et al. J. Am. Chem. Soc. 118:7461-7472, 1996; incorporated herein by reference), cRGDfK binds to αᵥβ₃ with high potency and selectivity. As the amino acid in position 5 has no influence on the activity, the primary amino group of the lysine in cRGDfK offers an ideal site for further functionalization of this cyclic peptide. Indeed, the amino group on the lysine side chain has been coupled with fluorescein, and the resulting conjugate was shown to be enriched in retina undergoing neovascularization (Hammes et al. Nature Med. 2:529-533, 1996; incorporated herein by reference).

The original synthesis of cRGDfK employed the Fmoc solid-phase chemistry to build a linear, protected RGDfK peptide, followed by cleavage from the solid resin, cyclization of the linear peptide, and removal of protecting groups (Haubner et al. J. Am. Chem. Soc. 118:7461-7472, 1996; incorporated herein by reference). Despite its high efficiency, several disadvantages remain. First, after the cleavage of the linear peptide from the resin, the cyclization of the acid terminal of glycine and the α-amino terminal of lysine was performed via in situ activation using diphenylphosphoryl azide (DPPA), requiring aqueous work-up. Second, the removal of the guanidine protecting group, 4-methoxy-2,3,6-trimethylbenzenesulfonyl (Mtr) group, from arginine necessitated the use of a mixture of several toxic and malodorous reagents, including trifluoroacetic acid (TFA), phenol, thioanisole, and ethanedithiol. And third, purification of the final product requires HPLC separation. Therefore, a simpler synthesis of cyclic RGD-containing peptides is needed that uses more environmentally friendly reagents and yields higher amounts of the purified product.

### SUMMARY OF THE INVENTION

The present invention provides a method and system for preparing cyclic peptides. According to the invention, a linear peptide is first synthesized or isolated using any method known in the art. The reactive groups on the peptide are preferably protected. The linear peptide is then cyclized so that an amide bond is formed between the carboxy terminus and the amino terminus of the peptide. The present method uses 1-propanephosphonic acid cyclic anhydride (T3P) for the cyclization of the linear peptide. This reagent is highly effective as a coupling reagent in peptide synthesis (Wissmann et al. Phosphorus Sulfur 30:645-648, 1987; Wissmann et al. Angew. Chem. 92:129-130; 1980; each of which is incorporated herein by reference). The product is then easily purified by silica gel chromatography, thereby eliminating the need for the toxic cyclizing reagent diphenylphosphoryl azide (DPPA). In a particularly preferred embodiment, for peptides containing arginine residues, the protecting group used to protect the arginine side chain is 2,2,5,7,8-pentamethylchroman-6-sulfonyl (Pbf), which requires mild deprotection conditions (water/trifluoroacetic acid (TFA): 1/19) (Fields et al. Tetrahedron Lett. 34:6661, 1993; Carpino et al. Tetrahedron Lett. 34:7782, 1993; each of which is incorporated herein by reference). An added advantage is that all other protecting groups can be removed using the same conditions required to remove the Pbf protecting group. The final deprotection step preferably does not involve the aforementioned toxic and high-boiling reagents and therefore renders unnecessary the purification of the final product by HPLC. To give but one example of the present method, the synthesis of the cyclic peptide cRGDfK is shown in FIG. 2.

### BRIEF DESCRIPTION OF THE DRAWING

FIG. 1 shows the RGD-containing peptides, cRGDfV and cRGDfK.

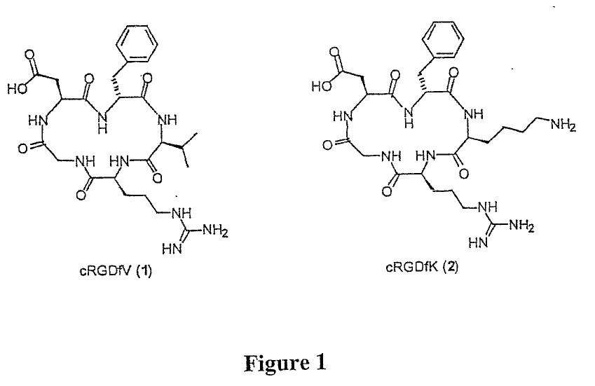

FIG. 2 shows the synthesis of cRGDfK using the inventive method.

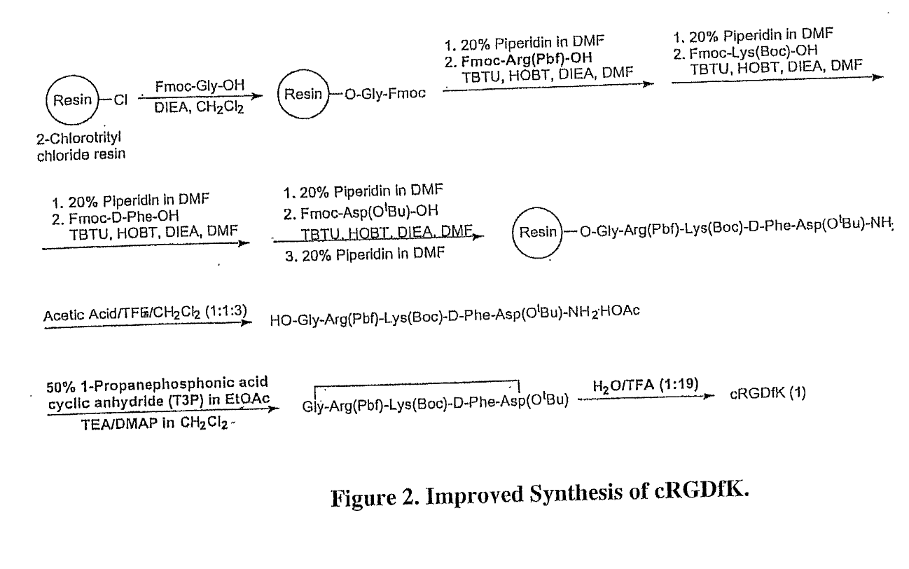

### DEFINITIONS

“Amino Acid”: According to the present invention, the term “amino acid” refers to any natural or unnatural amino acid. The amino acids may be of either the D or L configuration at the α carbon. In the present application, the lower case letter of the one letter abbreviation of the twenty natural amino acids is used to denote the D configuration at the α carbon. The amino acids may be further modified chemically (e.g., oxidation, reduction, esterification) or biologically (e.g., phosphorylation, hydroxylation).

“Cyclic”: A “cyclic” peptide is one in which one amino acid is covalently linked directly to another amino acid that is not found adjacent to it in the primary sequence of the linear peptide chain. In a preferred embodiment, the carboxy terminus of the peptide is linked to the amino terminus of the peptide. In another preferred embodiment, the covalent linkage between the two non-adjacent amino acids is an amide bond. For example, the linkage may be between the side chain of an aspartic acid residue and the amino terminus of the peptide, between the side chain of a lysine residue and the side chain of a glutamic acid residue, between the side chain of a lysine residue and the side chain of the carboxy terminus of the peptide, etc. In another preferred embodiment, the cyclic peptide results from the reaction of a linear peptide with 1-propanephosphonic acid cyclic anhydride (T3P).

“Peptide” or “Protein”: According to the present invention, a “peptide” or “protein” comprises a string of at least three amino acids linked together by peptide bonds. Inventive peptides preferably contain only natural amino acids, although non-natural amino acids (i.e., compounds that do not occur in nature but that can be incorporated into a polypeptide chain; see, for example, http://www.cco.caltech.edu/˜dadgrp/Unnatstruct.gif, which displays structures of non-natural amino acids that have been successfully incorporated into functional ion channels) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc.

### DETAILED DESCRIPTION OF CERTAIN PREFERRED EMBODIMENTS

The present invention provides a novel method of preparing cyclic peptides. Any cyclic peptide may be synthesized using this method. Preferably, the peptide comprises about 3-10 amino acids. In a preferred embodiment, the cyclic peptide is biologically active. In a particularly preferred embodiment, the cyclic peptide comprises the RGD motif found in inhibitors of the integrin family. Some particularly preferred sequences include RGDfK, RGDFK, RGDFV, RGDfV, RGDXX, RDGFX, and RGDfX (Please note that the lower case one-letter abbreviation represents the D-configuration of the amino acid rather than the naturally occurring L-configuration). In another preferred embodiment, the cyclic peptide to be synthesized is a naturally occurring, biologically active peptide, such as cylcosporin, bacitracin, colistin (polymixin E), polymixin B, or gramacidin S, or an analog thereof.

Peptides to be cyclized according to the present invention may be produced by any available method including but not limited to chemical or proteolytic cleavage of intact proteins or peptides, chemical synthesis, or in vitro or in vivo expression of an isolated or recombinant nucleic acid molecule. The peptide is preferably provided in purified form with the chemically reactive side chains protected.

In certain preferred embodiments, the peptides to be cyclized are prepared by chemical synthesis. Such peptides may utilize only naturally-occurring amino acids, or may include one or more non-natural amino acid analog or other chemical compound capable of being incorporated into a peptide chain. The linear peptide is preferably synthesized using standard solid phase peptide chemistry. In a particularly preferred embodiment, Fmoc solid phase chemistry is employed. In a preferred embodiment, the linear peptide is left with the side chains protected after cleavage from the resin, and the cyclization step using the reagent, 1-propanephosphonic acid cyclic anhydride, is performed with the side chains protected. After the cyclization is complete, the protecting groups are then removed. The cyclized, deprotected peptide is then optionally purified.

In synthesizing the peptide chemically, protecting groups may be used to mask chemically reactive moieties during certain steps. In a preferred embodiment, the protecting groups are chosen so that subjecting the peptide to one set of deprotection conditions will remove all protecting groups. In another preferred emobodiment, the deprotection conditions do not require toxic or malodorous or environmentally unfriendly reagents (e.g., phenol, thiols, amines, etc.). In a particularly preferred embodiment, the synthesis of arginine-containing peptides such as RGD-containing peptides uses the guanidine-protecting group, 2,2,5,7,8-pentamethylchroman-6-sulfonyl (Pbf) group.

After the peptide has been synthesized, it may be optionally purified. Any method known in the art may be used to purify the peptide at any stage in the synthesis. Techniques include but are not limited to crystallization, precipitation, extraction, affinity chromatography, silica gel chromatography, HPLC, reverse phase HPLC, FPLC, ion exchange chromatography, and gel filtration chromatography.

One particularly preferred method for preparing cyclic peptides according to the present invention is described below in the Example, which sets forth a multi-gram scale solid-phase synthesis of the cRGDfK. In comparison with the original synthesis of this compound, which has been reported in the literature, the inventive procedure not only eliminates the use of several toxic reagents, but also significantly improves the yield of the synthesis. These improvements are applicable to practical, large-scale synthesis of cRGDfV and other cyclic peptides.

These and other aspects of the present invention will be further appreciated upon consideration of the following Examples, which are intended to illustrate certain particular embodiments of the invention but are not intended to limit its scope, as defined by the claims.

### EXAMPLE

The synthesis of the linear peptide started with the incorporation of glycine onto o-chlorotrityl chloride resin by mixing the resin (16.72 g, 1.35 mmol/g, 22.57 mmol) with a solution of Fmoc-Gly-OH (16.78 g, 56.44 mmol) and N,N-diisopropylethylamine (DIEA, 9.04 mL, 51.90 mmol) in dry dichloromethane (DCM) in a 500 mL peptide synthesis vessel (ChemGlass). After shaking the reaction mixture for 2.5 hours using a Burrell Wrist-Action shaker, 9.0 mL of DIEA and 50 mL of methanol were added to cap the unreacted sites of the resin. After 30 minutes, the resin was washed with dimethylformamide (DMF, 2×), DCM (2×), methanol (2×), and diethylether (2×) and dried in vacuo. The loading of the resin was calculated as 17.60 mmol Fmoc-glycine. The Fmoc protecting group was then removed by shaking with a 20% solution of piperidine in DMF twice. The deprotection reaction was monitored by ninhydrin test. The deprotected resin is washed with DMF six times.

To the resin was added a solution of Fmoc-Arg(Pbf)-OH acids (25.00 g, 38.53 mmol), 2-(1H-benzotriazol-1-yl)-1,1,3,3-tetramethyluronium tetrafluoroborate (TBTU, 10.16 g, 31.64 mmol), 1-hydroxybenzotriazole (HOBt, 4.28 g, 31.67 mmol), and DIEA (19.68 mL, 112.98 mmol) in DMF. The mixture was mixed by shaking for 1.5 hr. The resulting resin was washed with DMF six times, deprotected by shaking with a 20% solution of piperidine in DMF twice, and washed with DMF for an additional six times. Subsequently, Fmoc-Lys(Boc)-OH, Fmoc-D-Phe-OH, and Fmoc-Asp(O^{t}Bu)-OH were coupled to the resin in the same manner.

The linear RGDfk peptide was cleaved from the resin without affecting other protecting groups by treating with 250 mL of a mixture of acetic acid, 2,2,2-trifluoroethane (TFE), and DCM (1:1:3) for one hour at room temperature. The resin was washed twice with 250 mL of the same mixture and then with DCM three times. The eluents were combined and concentrated. The excess acetic acid was azeotroped off with toluene.

The head-to-tail cyclization was performed by slowly adding the solution of the linear peptide acetate salt in 200 mL of DCM to a solution of 1-propanephosphonic acid cyclic anhydride (T3P, 54.24 mL), triethyl amine (TEA, 63.2 mL), and DMAP (200 mg) in 8 liter of DCM. After stirring overnight, the starting material could not be detected by RP-HPLC. The reaction mixture was concentrated, purefied by flash chromatography (methanol/ethyl acetate, 1:10) to afford 17.23 g (96.7%, 17.02 mmol, relative to the amount of Gly coupled to the resin) of the protected cyclic peptide as light yellow solid (MW=1012.23).

The protecting groups of the above cyclic peptide (16.05 g) were removed with a mixture of water and trifluoroacetic acid (TFA) (1/19). TFA was removed by azotroping with toluene. Trituration of the DCM solution of the product in diethyl ether followed by filtration yield cRGDfK.2 TFA salt (MW=831.72, 10.81 g, 82.0%) as light yellow powder. The overall yield (79.3% relative to the amount of Gly coupled to the resin) is significantly higher than the reported yield (44%) (Haubner et al. J. Am. Chem. Soc. 118:7461-7472, 1996; incorporated herein by reference). The cyclic peptide salt obtained this way is pure and who only one peak in reversed phase high-performance liquid chromatography (RP-HPLC). ESI mass spectrum and NMR of the compound were identical to those previously reported (Haubner et al. J. Am. Chem. Soc. 118:7461-7472, 1996; incorporated herein by reference).

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

What is claimed is:

1. A method of synthesizing cyclic peptides, the method comprising steps of:
providing a linear peptide; and
cyclizing the linear peptide.

2. The method of claim 1 wherein the linear peptide comprises a sequence of RGDFK.

3. The method of claim 1 wherein the linear peptide comprises a sequence of RGDfK.

4. The method of claim 1 wherein the linear peptide comprises a sequence of RGDFV.

5. The method of claim 1 wherein the linear peptide comprises a sequence of RGDfV.

6. The method of claim 1 wherein the linear peptide comprises a sequence selected from the group consisting of RGDXX, RGDFX, and RGDfX.

7. The method of claim 1 wherein the linear peptide comprises an arginine (R) residue.

8. The method of claim 1 wherein the linear peptide comprises 2-10 amino acids.

9. The method of claim 1 wherein the step of cyclizing comprises treating the linear peptide with 1-propanephosphonic acid cyclic anhydride (T3P).

10. A method of synthesizing cyclic peptides, the method comprising steps of:
providing an arginine containing peptide wherein the arginine side chain is protected with 2,2,5,7,8-pentamethylchroman-6-sulfonyl (Pbf); and
cyclizing the peptide using 1-propanephosphonic acid cyclic anhydride (T3P).

11. The method of claim 10 wherein the method comprises the additional step of:
deprotecting the peptide.

12. The method of claim 10 wherein the method comprises the additional step of:
purifying the peptide.
